(19) United States
(12) Patent Application Publication
(10) Pub. No.: US 2025/0108083 A1
(43) Pub. Date: Apr. 3, 2025

(54) HERBAL FORMULATIONS, ITS METHOD OF PREPARATION AND ITS METHOD OF USE

(71) Applicants: Nanyang Technological University, Singapore (SG); Nanyang Herbs Pte. Ltd., Singapore (SG)

(72) Inventors: Hoi Yeung LI, Singapore (SG); Cheng Gee KOH, Singapore (SG); Soak Kuan LAI, Singapore (SG); Siu Cheung Eddie SO, Singapore (SG); Toon Wah Roland ONG, Singapore (SG)

(21) Appl. No.: 18/730,208

(22) PCT Filed: Feb. 10, 2023
(86) PCT No.: PCT/SG2023/050073

§ 371 Date: Jul. 18, 2024

(30) Foreign Application Priority Data
Feb. 11, 2022 (SG) 10202201347R

(51) Int. Cl. A61K 36/28; A61K 36/19; A61K 36/195; A61K 36/53; A61K 36/87; A61P 35/00

(52) U.S. Cl. CPC A61K 36/28; A61K 36/19; A61K 36/195; A61K 36/53; A61K 36/87; A61P 35/00; A61K 2236/333; A61K 2236/53; A61K 2236/55

(57) ABSTRACT

Disclosed herein is a herbal formulation comprising a herbal extract of Elephantopus tomentosus L and a herbal extract of Leea indica, which may be used in the treatment of cancer (e.g. colon cancer). The herbal formulation may further comprise one or more of: an extract of Clinacanthus nutans; an extract of Strobilanthes crispus L; and an extract of Callicarpa pedunculata R. Br.

### FIELD OF INVENTION

This invention provides herbal formulations, their preparation and their use as a therapy and/or as an adjuvant therapy to standard treatment for colon cancer.

### BACKGROUND

The listing or discussion of a prior-published document in this specification should not necessarily be taken as an acknowledgement that the document is part of the state of the art or is common general knowledge.

Cancer is one of the leading causes of death globally and the morbidity associated with this disease has been increasing. This highly complex disease makes current cancer treatment strategies, which include surgery, chemotherapy, radiotherapy, gene and immunotherapy, inadequate. None of these treatment methods are able to achieve optimal curative effect due to drug resistance and adverse side effects. Therefore, there is a need to discover and develop alternative therapy options for cancer prevention and treatment.

Traditional prescriptions involving herbal formulations have been used extensively for thousands of years to treat wounds and diseases. These prescriptions have often been put in doubt and questioned by physicians in the past on the quality and consistency, as well as the lack of scientific basis and evidence from clinical trials. Nevertheless, herbal medicines are now becoming increasingly accepted and are used as a form of complementary and alternative treatment for cancer. The conventional approach of single drug-single target has proven insufficient and limited in their effectiveness to treat complex chronic diseases, resulting in a shift towards adopting multi-drug or multi-component therapies. The use of traditional herbal medicines, which typically utilizes complex herbal formulations, allows for complex interactions between compounds/components within a herb or multiple herbs to act in a synergistic, antagonistic, addictive or potentiated manner that is favourable to match the complexity of cancer. Numerous studies have found that herbal medicines are able to modulate the body system in a more holistic way, in which they are able to help strengthen the patient's immune system and control the side effects and toxicities arising from mainstream cancer therapies. Consequently, this helps to improve the patient's chance of recovery, quality of life and prevent recurrence.

Patents have been granted in the United States for herbal formulations that are being used for cancer treatment or cancer-related health problems (Feng, Y. et al., Recent Pat. Food Nutr. Agric. 2011, 3, 30-48). Furthermore, several herbal formulations have demonstrated beneficial effects in preclinical or clinical trials—for example, Bu-Zhong-Yi-Qi-Tang (TJ-41), Shi-Quan-Da-Bu-Tang (TJ-48), Xiao-chai-hu-tang (TJ-9), and more notably, PHY906 (Wang, Z. et al., Biosci. Trends 2018, 12, 220-239). However, none of these formulations have yet gotten FDA approval to become a prescription medicine.

Therefore, there exists a need for new herbal formulations that can be used as a medicine or supplement for cancer treatment, prevention or serve as an adjuvant therapy for standard treatment.

### SUMMARY OF INVENTION

1. A herbal formulation comprising a herbal extract of Elephanopus tomentosus L and a herbal extract of Leea indica.

2. The herbal formulation according to Clause 1, wherein the formulation comprises one or more of:
an extract of Clinacanthus nutans;
an extract of Strobilanthes crispus L.; and
an extract of Callicarpa pedunculata R. Br.

3. The herbal formulation according to Clause 1, wherein the formulation consists of the herbal extract of Elephanopus tomentosus L and the herbal extract of Leea indica.

4. The herbal formulation according to any one of the preceding clauses, wherein the weight to weight ratio of the herbal extract of Elephanopus tomentosus L and the herbal extract of Leea indica is from 1:1 to 5:1, such as about 1.7:1

5. The herbal formulation according to any one of Clauses 2 and 3 to 4, as dependent upon Clause 2, wherein the extract of Clinacanthus nutans is present.

6. The herbal formulation according to Clause 5, wherein the extract of Strobilanthes crispus L. is also present.

7. The herbal formulation according to Clause 6, wherein the weight to weight ratio of the extract of Clinacanthus nutans and the extract of Strobilanthes crispus L. is from 1:0.4 to 1:2.

8. The herbal formulation according to any one of Clauses 5 to 7, wherein the weight to weight ratio of the extract of Clinacanthus nutans and the extract of Elephanopus tomentosus L. is from 1:1 to 1:11.

9. The herbal formulation according to any one of Clauses 5 to 8, wherein the weight to weight ratio of the extract of Clinacanthus nutans and the extract of Leea indica is from 1:6.5 to 1:8.

10. The herbal formulation according to any one of Clauses 5 to 9, wherein the extract of Callicarpa pedunculata R. Br. is also present.

11. The herbal formulation according to Clause 6, wherein the weight to weight ratio of the extract of Clinacanthus nutans and the extract of Callicarpa pedunculata R. Br. is from 1:1.5 to 1:2.

12. The herbal formulation according to any one of Clauses 2 and 3 to 11, as dependent upon Clause 2, wherein the formulation comprises:
the extract of Clinacanthus nutans;
the extract of Strobilanthes crispus L.;
the extract of Elephanopus tomentosus L.;
the extract of Leea indica; and
the extract of Callicarpa pedunculata R. Br.

13. The herbal formulation according to Clause 12, wherein the weight to weight ratio of the extract of Clinacanthus nutans to the extract of Strobilanthes crispus L. to the extract of Elephanopus tomentosus L. to the extract of Leea indica to the extract of Callicarpa pedunculata R. Br. is 2:1:22:13:4.

14. The herbal formulation according to any one of the preceding clauses, wherein each extract is obtained from a solvent comprising an organic solvent, optionally wherein the organic solvent is ethanol, further optionally wherein the ethanol in the solvent is about 65 vol % of the solvent.

15. A method of preparing a herbal formulation, comprising:
(a) providing stock solutions of:
   an extract of Elephanopus tomentosus L.; and
   an extract of Leea indica; and
(b) combining a portion of the stock solution of the extract of Elephanopus tomentosus L. with a portion of the stock solution of the extract of Leea indica to provide the herbal formulation.

16. The method according to Clause 15, wherein the method further comprises:
(i) further providing stock solutions of:
   an extract of Clinacanthus nutans;
   an extract of Strobilanthes crispus L.; and
   an extract of Callicarpa pedunculata R. Br; and
(ii) combining a portion of one or more of the extract of Clinacanthus nutans, the extract of Strobilanthes crispus L. and the extract of Callicarpa pedunculata R. Br in step (b) of the method according to Clause 15.

17. The method according to Clause 15 or Clause 16, wherein each extract is prepared by
(ai) providing a powder of a freeze-dried herb;
(aii) mixing the powder of a freeze-dried herb with a first solvent to provide a mixture and subjecting the mixture to sonication for a period of time to provide a sonicated mixture;
(aiii) removing solids from the sonicated mixture by centrifugation to collect a soluble portion of the sonicated mixture; and
(aiv) removing the solvent from the soluble portion and then adding a second solvent to provide a stock solution, optionally wherein the stock solution has a concentration of from 50 to 200 mg/mL, such as about 100 mg/mL

18. The method according to any one of Clauses 15 to 17, wherein the first solvent comprises an organic solvent, optionally wherein the organic solvent is ethanol, optionally wherein the ethanol in the first solvent is about 65 vol % of the first solvent.

19. The method according to any one of Clauses 16 to 18, wherein the method further comprises a step of removing the solvent to provide a pellet.

20. A method of preparing a herbal formulation, comprising:
(bi) providing a powder of two of more freeze-dried herbs;
(bii) mixing the powder of two or more freeze-dried herbs with a first solvent to provide a mixture and subjecting the mixture to sonication for a period of time to provide a sonicated mixture;
(biii) removing solids from the sonicated mixture by centrifugation to collect a soluble portion of the sonicated mixture as the herbal formulation, wherein
   the two or more herbs comprise Elephanopus tomentosus L. and Leea indica.

21. The method according to Clause 20, wherein the two or more herbs further comprise one or more of Clinacanthus nutans, Strobilanthes crispus L. and Callicarpa pedunculata R. Br.

22. The method according to Clause 20 or Clause 21, wherein the method further comprises:
(biv) removing the solvent from the soluble portion to provide the herbal formulation in a pellet form.

23. The method according to any one of Clauses 20 to 22, wherein the first solvent comprises an organic solvent, optionally wherein the organic solvent is ethanol, optionally wherein the ethanol in the first solvent is about 65 vol % of the first solvent.

24. A pharmaceutical composition comprising a herbal formulation as defined in any one of Clauses 1 to 14 and a pharmaceutically acceptable excipient.

25. Use of a herbal formulation as defined in any one of Clauses 1 to 14 in the preparation of a medicament for the treatment of cancer.

26. A herbal formulation as defined in any one of Clauses 1 to 14, for use in the treatment of cancer.

27. A method of treating cancer, which method comprises administering a therapeutically effective amount of a herbal formulation as defined in any one of Clauses 1 to 14 to a subject in need thereof.

28. The use according to Clause 20, the herbal formulation for use according to Clause 21, or the method according to Clause 22, wherein the cancer is selected from one or more of the group selected from adrenal cancer, anal cancer, bile duct cancer, bladder cancer, bone cancer, brain tumours, CNS tumours, breast cancer, Castleman disease, cervical cancer, colon cancer, rectum cancer, colorectal cancer, endometrial cancer, esophagus cancer, eye cancer, gallbladder cancer, gastrointestinal carcinoid tumors, gastric cancer, gastrointestinal stromal tumor (GIST), gestational trophoblastic disease, Hodgkin disease, Kaposi sarcoma, kidney cancer, laryngeal cancer, hypopharyngeal cancer, leukemia (e.g. acute lymphocytic, acute myeloid, chronic lymphocytic, chronic myeloid, chronic myelomonocytic), liver cancer, lung cancer (e.g. small cell or non-small cell), lung carcinoid tumour, lymphoma (e.g. of the skin), malignant mesothelioma, multiple myeloma, myelodysplastic syndrome, nasal cavity cancer, paranasal sinus cancer, nasopharyngeal cancer, neuroblastoma, non-Hodgkin lymphoma, oral cavity cancer, oropharyngeal cancer, osteosarcoma, ovarian cancer, pancreatic cancer, penile cancer, pituitary tumours, prostate cancer, retinoblastoma, rhabdomyosarcoma, salivary gland cancer, sarcoma, skin cancer (basal and squamous cell, melanoma, Merkel cell), small intestine cancer, stomach cancer, testicular cancer, thymus cancer, thyroid cancer, uterine sarcoma, vaginal cancer, vulvar cancer, Waldenstrom macroglobulinemia, Wilms tumour.

29. The use, compound for use or method according to Clause 23, wherein the cancer is colon cancer.

### DRAWINGS

FIG. 1 depicts the IC₅₀ curves of the individual herb extracts and formulations on the viability of colon cancer cells.

FIG. 2 depicts the herb treatment limits DLD-1 clonogenicity. (A) Representative images of DLD-1 colonies formed 1 week after cells were treated for 1 hour with the various herb extracts at their respective low (LD), medium (MD) or high dose (HD) concentrations, or left untreated to serve as a control. (B) Colony counts and percentages of well areas occupied by colonies formed by DLD-1 cells treated with herb extracts at their respective LD, MD, or HD concentrations compared with untreated control colonies. Data represents mean±standard deviation (n=3). *=p≤0.05, **=p<0.01, ***=p≤0.001, ****=p≤0.0001.

FIG. 3 depicts that the growth of DLD-1 spheroids is inhibited by herb treatment. (A) Representative phase-contrast images of three-dimensional DLD-1 tumour spheroids captured on Days 0 and 15 of treatment with the various herb extracts at their respective LD, MD or HD concentrations, or left untreated to serve as a control. Scale bar=250 μm. (B) Spheroid growth curves and endpoint spheroid areas after 15 days of treatment with herb extracts at their respective LD, MD, or HD concentrations compared with untreated control spheroids. All measurements exclude areas occupied by non-spheroid forming cells. Data represents mean±standard deviation (n=3). *=p≤0.05, **=p≤0.01.

FIG. 4 depicts the live imaging of untreated DLD-1 cells, or incubated with LD, MD, or HD concentrations of Herb 1, Herb 2, Herb 3, Herb 4, Herb 5 and formulations H34 and H12345 for 72 hours. Scale bar=100 μm.

FIG. 5 depicts that the cell cycle distribution is altered in herb-treated DLD-1 cells. (A) Representative cell cycle histograms of DLD-1 treated for 24 hours with H3, H34 and H12345 at their respective LD, MD, or HD concentrations, or left untreated to serve as a control. (B) Distribution of post-treatment population across cell cycle stages by treatment group and dose. (C-E) Comparison of cell cycle distribution between doses within treatment groups. Data represents mean±standard deviation (n=3). *=p<0.05, **=p≤0.01, ***=p≤0.001.

FIG. 6 depicts the dose dependent effect on herb extracts treatment on the expression of pro-apoptotic proteins in DLD-1 cells. Untreated cells served as control. β-tubulin acted as a loading control. Results shown were consistent over at least three independent experiments.

FIG. 7 depicts that the herbal formulation H34 can reduce tumour growth in HCT116 and DLD-1 xenografted mice models. Tumour volume (mm³) of the (A) HCT116 and (B) DLD-1 xenograft model during the course of treatment. (C) Example of tumour size comparison between control normal mice and mice fed with H34 formulation at experimental endpoint in DLD-1 xenografted model. Measurement of tumour volumes (mm³) and tumour weight (g) dissected from (D, E) HCT116 xenograft mice model and (F, G) DLD-1 xenograft mice model. **p<0.05, 1 tail T-test, 95% confidence; *p<0.1, 1 tail T-test, 90% confidence.

FIG. 8 depicts the evaluation of tumour cell growth in HCT116 xenografted mice model treated with H12345 formulation. (A) Tumour volume (mm³) of the HCT116 xenograft model during the course of treatment. (B) Example of tumour size comparison between control normal mice and mice fed with H12345 formulation at experimental endpoint in HCT116 xenografted model. (C, D) Measurement of tumour volumes (mm³) and tumour weight (g) dissected from HCT116 xenograft mice model. **p<0.05, 1 tail T-test, 95% confidence.

### DESCRIPTION

It has been surprisingly found that a combination of specific herbal extracts can be used to treat cancer. Thus, in a first aspect of the invention, there is provided a herbal formulation comprising a herbal extract of Elephanopus tomentosus L and a herbal extract of Leea indica.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

In certain embodiments of the invention, the the formulation may further comprise one or more of:
an extract of Clinacanthus nutans;
an extract of Strobilanthes crispus L.; and
an extract of Callicarpa pedunculata R. Br.

Clinacanthus nutans, better known as Sabah snake grass, is a species of the Acanthaceae family and native to Southeast Asia regions. Claims about its anticancer properties usually come from in vitro studies using a range of cancer cell lines and primarily the leaves of the plants which is subjected to different extraction methods. Varying levels of cytotoxicity have been reported in the different cell lines with little or no detailed information about mechanistic actions and animal study (Alam, A. et al., Asian Pac. J. Trop. Med. 2016, 9, 402-409; and Khoo, L. W. et al., Evid. Based Complement. Alternat. Med. 2018, 2018, 9276260).

Strobilanthes crispus L., also known as Black Face General, is traditionally used as a remedy for many ailments including cancer. The leaves of the plant are commercially available as herbal tea product but the scientific basis behind the use is undetermined. Studies have shown that the leaf extracts can kill breast and prostate cancer cells but non-cytotoxic to non-cancerous breast epithelial cells (Yaacob, N. S. et al., BMC Complement. Altern. Med. 2010, 10, 42).

Elephantopus tomentosus Linn. is a species of perennial flowering plant belonging to the Asteraceae family. It is native to North America but has spread widely to the pantropical regions. A bulk of the pharmacological studies involves bioactive compounds isolated from the plant and several of these compounds have demonstrated cancer cell cytotoxicity and antitumor efficacy (Kabiru, A. & Por, L. Y., Advances in Life Science and Technology 2013, 15, 6-13; Wang, B. et al., Chin. J. Chem. 2012, 30, 1320-1322; and Hayashi, T. et a., J. Nat. Prod. 1999, 62, 302-304).

Leea indica (Burm. f Merr.) is a perennial shrub which can be found in the tropical or subtropical countries. The leaves are usually eaten raw or taken as a concoction brewed from fresh leaves to treat various ailments. The antioxidant and anticancer activity of the Leea indica leaves have been shown on some prostate and cervical cell lines (Ghagane, S. C. et al., Integr. Med. Res. 2017, 6, 79-87; and Wong, Y. H. & H. A. Kadir, Evid. Based Complement. Alternat. Med. 2011, 2011, 293060).

Callicarpa pedunculata R. Br is a shrub or small tree in the Lamiaceae family. Some species from the Callicarpa genus have reported to be used against cancer but there is no report about the anticancer activity of Callicarpa pedunculata R. Br (Jones, W. P. & Kinghorn, A. D., Curr. Bioact. Compd. 2008, 4, 15-32).

In particular embodiments of the invention, the herbal formulation may be one that consists of the herbal extract of Elephanopus tomentosus L and the herbal extract of Leea indica. In such formulations, any suitable weight to weight ratio of the two herb extracts may be used, and this may be determined by a skilled person. For example, the weight to weight ratio of the herbal extract of Elephanopus tomentosus L and the herbal extract of Leea indica may be from 1:1 to 5:1, such as about 1.7:1

In certain embodiments that may be mentioned herein, the herbal formulation may also include the extract of Clinacanthus nutans. In such embodiments, the herbal formulation may also include the extract of Strobilanthes crispus L. In such embodiments, any suitable weight to weight ratio of the extract of Clinacanthus nutans and the extract of Strobilanthes crispus L. may be used herein. For example, the weight to weight ratio of the extract of Clinacanthus nutans and the extract of Strobilanthes crispus L. may be from 1:0.4 to 1:2. Additionally or alternatively, any suitable weight to weight ratio of the extract of Clinacanthus nutans and the extract of Elephanopus tomentosus L. may be used herein. For example, the weight to weight ratio of the extract of Clinacanthus nutans and the extract of Elephanopus tomentosus L. may be from 1:1 to 1:11. Additionally or alternatively, any suitable weight to weight ratio of the extract of Clinacanthus nutans and the extract of Leea indica may be used herein. For example, the weight to weight ratio of the extract of Clinacanthus nutans and the extract of Leea indica may be from 1:6.5 to 1:8.

In certain embodiments that may be mentioned herein, the formulation may further comprise the extract of Callicarpa pedunculata R. Br. any suitable weight to weight ratio of the extract of Clinacanthus nutans and the extract of Callicarpa pedunculata R. Br. may be used herein. For example, the weight to weight ratio of the extract of Clinacanthus nutans and the extract of Callicarpa pedunculata R. Br. may be from 1:1.5 to 1:2.

In particular embodiments of the invention, the herbal formulation may be one where the formulation comprises:
the extract of Clinacanthus nutans;
the extract of Strobilanthes crispus L.;
the extract of Elephanopus tomentosus L.;
the extract of Leea indica; and
the extract of Callicarpa pedunculata R. Br.

Any suitable weight to weight ratios of the components mentioned above may be used herein (e.g. as indicated hereinbefore). For example, the weight to weight ratio of the extract of Clinacanthus nutans to the extract of Strobilanthes crispus L. to the extract of Elephanopus tomentosus L. to the extract of Leea indica to the extract of Callicarpa pedunculata R. Br. may be 2:1:22:13:4.

In embodiments of the invention, each herbal extract mentioned hereinbefore may be obtained through extraction with a solvent that comprises a suitable organic solvent. An example of a suitable organic solvent includes, but is not limited to ethanol. For example, the solvent may be about 65 vol % ethanol. The remainder of the solvent may be water.

The herbal formulation may be obtained by providing stock solutions of the extracts and combining them together, followed by optionally removing the solvent. Alternatively, each of the desired herbs may be combined and extracted together to provide the extract, following which the solvent may be removed.

Thus in a further aspect of the invention, there is provided a method of preparing a herbal formulation, comprising:
(a) providing stock solutions of:
   an extract of Elephanopus tomentosus L.; and
   an extract of Leea indica; and
(b) combining a portion of the stock solution of the extract of Elephanopus tomentosus L. with a portion of the stock solution of the extract of Leea indica to provide the herbal formulation.

As noted above, the formulations may also include additional herb extracts. Thus, the method above may further comprise:
(i) further providing stock solutions of:
   an extract of Clinacanthus nutans;
   an extract of Strobilanthes crispus L.; and
   an extract of Callicarpa pedunculata R. Br; and
(ii) combining a portion of one or more of the extract of Clinacanthus nutans, the extract of Strobilanthes crispus L. and the extract of Callicarpa pedunculata R. Br in step (b) of the method according to Claim 15.

In the above embodiments, each of the extracts may be prepared by:
(ai) providing a powder of a freeze-dried herb;
(aii) mixing the powder of a freeze-dried herb with a first solvent to provide a mixture and subjecting the mixture to sonication for a period of time to provide a sonicated mixture;
(aiii) removing solids from the sonicated mixture by centrifugation to collect a soluble portion of the sonicated mixture; and
(aiv) removing the solvent from the soluble portion and then adding a second solvent to provide a stock solution, optionally wherein the stock solution has a concentration of from 50 to 200 mg/mL, such as about 100 mg/mL

As will be appreciated, the solvent may be removed from the formulation to provide a dried form, which may be in the form of a pellet. Thus, the method may further comprise a step of removing the solvent to provide a pellet.

In embodiments of the invention, each herbal extract mentioned hereinbefore may be obtained through extraction with a solvent that comprises a suitable organic solvent. An example of a suitable organic solvent includes, but is not limited to ethanol. For example, the solvent may be about 65 vol % ethanol. The remainder of the solvent may be water.

In a further aspect of the invention, there is provided a method of preparing a herbal formulation, comprising:
(bi) providing a powder of two of more freeze-dried herbs;
(bii) mixing the powder of two or more freeze-dried herbs with a first solvent to provide a mixture and subjecting the mixture to sonication for a period of time to provide a sonicated mixture;
(biii) removing solids from the sonicated mixture by centrifugation to collect a soluble portion of the sonicated mixture as the herbal formulation, wherein
   the two or more herbs comprise Elephanopus tomentosus L. and Leea indica.

As will be appreciated, the herbal formulation may further comprise one or more of Clinacanthus nutans, Strobilanthes crispus L. and Callicarpa pedunculata R. Br. As such, the two of more freeze-dried herbs may also comprise one or more of these additional herbs too.

As will be appreciated, the solvent may be removed from the formulation to provide a dried form, which may be in the form of a pellet. Thus, the method may further comprise a step of removing the solvent to provide a pellet.

In embodiments of the invention, each herbal extract mentioned hereinbefore may be obtained through extraction with a solvent that comprises a suitable organic solvent. An example of a suitable organic solvent includes, but is not limited to ethanol. For example, the solvent may be about 65 vol % ethanol. The remainder of the solvent may be water.

In a further aspect of the invention, there is provided a pharmaceutical composition comprising a herbal formulation as described hereinbefore and a pharmaceutically acceptable excipient.

The herbal formulations and pharmaceutical compositions disclosed herein may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Particular modes of administration that may be mentioned include oral, intravenous, cutaneous, subcutaneous, nasal, intramuscular or intraperitoneal administration.

The herbal formulations disclosed herein will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of the herbal formulation disclosed herein in any pharmaceutical formulation used in accordance with the present invention will depend on various factors, such as the severity of the condition to be treated, the particular patient to be treated, as well as the compound(s) which is/are employed. In any event, the amount of compound of formula I in the formulation may be determined routinely by the skilled person.

For example, a solid oral composition such as a tablet or capsule may contain from 1 to 99% (w/w) active ingredient (i.e. the herbal formulation); from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient (i.e. the herbal formulation); and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, the herbal formulation may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula I.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

As noted hereinbefore the herbal formulation may be useful in the treatment of cancer. Thus, in yet further aspects of the invention, there is:
(A) use of a herbal formulation as described hereinbefore in the preparation of a medicament for the treatment of cancer;
(B) a herbal formulation as described hereinbefore, for use in the treatment of cancer; and
(C) a method of treating cancer, which method comprises administering a therapeutically effective amount of a herbal formulation as described hereinbefore to a subject in need thereof.

The herbal formulation may be used to treat any suitable cancer. For example, the cancer may be selected from one or more of the group selected from adrenal cancer, anal cancer, bile duct cancer, bladder cancer, bone cancer, brain tumours, CNS tumours, breast cancer, Castleman disease, cervical cancer, colon cancer, rectum cancer, colorectal cancer, endometrial cancer, esophagus cancer, eye cancer, gallbladder cancer, gastrointestinal carcinoid tumors, gastric cancer, gastrointestinal stromal tumor (GIST), gestational trophoblastic disease, Hodgkin disease, Kaposi sarcoma, kidney cancer, laryngeal cancer, hypopharyngeal cancer, leukemia (e.g. acute lymphocytic, acute myeloid, chronic lymphocytic, chronic myeloid, chronic myelomonocytic), liver cancer, lung cancer (e.g. small cell or non-small cell), lung carcinoid tumour, lymphoma (e.g. of the skin), malignant mesothelioma, multiple myeloma, myelodysplastic syndrome, nasal cavity cancer, paranasal sinus cancer, nasopharyngeal cancer, neuroblastoma, non-Hodgkin lymphoma, oral cavity cancer, oropharyngeal cancer, osteosarcoma, ovarian cancer, pancreatic cancer, penile cancer, pituitary tumours, prostate cancer, retinoblastoma, rhabdomyosarcoma, salivary gland cancer, sarcoma, skin cancer (basal and squamous cell, melanoma, Merkel cell), small intestine cancer, stomach cancer, testicular cancer, thymus cancer, thyroid cancer, uterine sarcoma, vaginal cancer, vulvar cancer, Waldenstrom macroglobulinemia, Wilms tumour. In particular embodiments of the invention, the cancer may be colon cancer.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

The aspects of the invention described herein (e.g. the above-mentioned compounds, combinations, methods and uses) may have the advantage that, in the treatment of the conditions described herein, they may be more convenient for the physician and/or patient than, be more efficacious than, be less toxic than, have better selectivity over, have a broader range of activity than, be more potent than, produce fewer side effects than, or may have other useful pharmacological properties over, similar compounds, combinations, methods (treatments) or uses known in the prior art for use in the treatment of those conditions or otherwise.

As demonstrated in the examples below, the herbal formulations disclosed herein (e.g. H34 and H12345), have been shown to be effective in inhibiting the cell viability of colon cancer cells in vitro and also demonstrated significant anti-tumour cell growth in vivo. There is a strong potential that these formulations can eventually be used in colon cancer treatment or can serve as adjuvants in conventional cancer therapies.

In a further aspect of the invention, there is provided a herbal formulation for the treatment of cancer, the herbal formulation comprising effective amount of at least two of the following: Clinacanthus nutans extract (H1), Strobilanthes crispus L. extract (H2), Elephanopus tomentosus L. extract (H3), Leea indica extract (H4) and Callicarpa pedunculata R. Br extract (H5).

In various embodiments, the herbal formulation comprises an effective amount of Elephanopus tomentosus L. extract (H3) and Leea indica extract (H4). In such embodiments, the Elephanopus tomentosus L. extract (H3) and the Leea indica (H4) extract are mixed in weighted ratio of at least 1:1 but not more than 5:1 respectively. For example, the Elephanopus tomentosus L. extract (H3) and the Leea indica extract (H4) are mixed in weighted ratio of 1.7:1 respectively.

In various embodiments, the herbal formulation comprises an effective amount of Clinacanthus nutans extract (H1) and Strobilanthes crispus L. extract (H2). In such embodiments, the Clinacanthus nutans extract (H1) and the Strobilanthes crispus L. extract (H2) are mixed in weighted ratio ranging from 1:0.4 to 1:2 respectively.

In various embodiments, the herbal formulation comprises an effective amount of Clinacanthus nutans extract (H1) and Elephanopus tomentosus L. extract (H3). In such embodiments, the Clinacanthus nutans extract (H1) and Elephanopus tomentosus L. extract (H3) are mixed in weighted ratio ranging from 1:1 to 1:11 respectively.

In various embodiments, the herbal formulation comprises an effective amount of Clinacanthus nutans extract (H1) and Leea indica extract (H4). In such embodiments, the Clinacanthus nutans extract (H1) and the Leea indica extract (H4) are mixed in weighted ratio ranging from 1:6.5 to 1:8 respectively.

In various embodiments, the herbal formulation comprises an effective amount of Clinacanthus nutans extract (H1) and Callicarpa pedunculata R. Br extract (H5). In such embodiments, the Clinacanthus nutans extract (H1) and the Callicarpa pedunculata R. Br extract (H5) are mixed in weighted ratio ranging from 1:1.5 to 1:2 respectively.

In various embodiments, the herbal formulation comprises an effective amount of Clinacanthus nutans extract (H1), Strobilanthes crispus L. extract (H2), Elephanopus tomentosus L. extract (H3), Leea indica extract (H4) and Callicarpa pedunculata R. Br extract (H5). In such embodiments, the Clinacanthus nutans extract (H1), Strobilanthes crispus L. extract (H2), Elephanopus tomentosus L. extract (H3), Leea indica extract (H4) and Callicarpa pedunculata R. Br extract (H5) are mixed in weighted ratio of 2:1:22:13:4 respectively.

In a further aspect of the invention, there is provided use of the herbal formulation described herein in the treatment of cancer.

In various embodiments, the cancer may be colon cancer.

Further aspects and embodiments of the invention will now be discussed by reference to the following non-limiting examples.

### EXAMPLES

### Materials

Raw herbs (Clinacanthus nutans, Strobilanthes crispus L., Elephanopus tomentosus L., Leea indica and Callicarpa pedunculata R. Br) were obtained from the Nanyang Community Herb Garden located at Nanyang Technological University. Human colorectal adenocarcinoma cells (DLD-1) and colorectal carcinoma cells (HCT1116) were purchased from American Type Culture Collection (ATCC). Five to six-weeks old female J:Nu outbred nude mice were purchased from InVivos. Rosewell Park Memorial Institute (RPMI) 1640 medium was purchased from Gibco, USA. Fetal bovine serum (FBS), Penicillin and Streptomycin were purchased from GE Hyclone, USA. 0.45 μm polyvinylidene fluoride (PVDF) membranes were purchased from Bio-Rad, USA. WesternBright™ ECL horseradish peroxidase (HRP) Substrate kit was purchased from Advansta, USA. Dimethyl sulfoxide (DMSO), 3-(4,5-Dimethyl-2-thiazolyl)-2,5-diphenyl-2H-tetrazolium bromide (MTT), and ethanol were purchased from Sigma-Aldrich. B-tubulin antibody, crystal violet used in the clonogenic assay, agarose used in tumor spheroid assay, propidium iodide, RNaseA, and skimmed milk powder were purchased from Sigma Aldrich (USA). Caspase 3, cleaved caspase 3, cleaved caspase 7, and cleaved PARP1 antibodies were purchased from Cell Signaling (USA). Goat anti-mouse and anti-rabbit IgG HRP antibodies were purchased from Invitrogen (USA). Phosphate buffered saline (PBS) and tris-buffered saline with Tween 20 (TBST) were prepared in the lab.

### Example 1. Preparation of Herbal Extracts with 65% Ethanol as a Solvent

The leaves of the different plants were used as the raw material to make the herbal extracts. The leaves were rinsed to remove any dust and dirt before freeze drying. The ground freeze dried powder was forcefully mixed with thirty times its equivalent volume of 65% ethanol and then subjected to sonication for 30 minutes. This was followed by centrifugation at 4000 rpm for 15 minutes to remove the insoluble residue. The supernatant was evaporated to dryness and the pellet weight of ethanol soluble fraction was measured. The pellet was subsequently dissolved in water to give a 100 mg/ml stock solution. Herbal extracts were made individually from each plant first before combining them in a specific ratio to make the herbal formulations. Extracts derived from Clinacanthus nutans, Strobilanthes crispus L., Elephanopus tomentosus L., Leea indica and Callicarpa pedunculata R. Br are referred to as H1, H2, H3, H4 and H5, respectively, hereinafter. The two herbs formulation comprises H3 and H4 and has a weighted ratio of at least 1:1 but not more than 5:1 and is referred to as H34 formulation hereinafter. The five herbs formulation comprises of H1, H2, H3, H4 and H5, wherein the weighed ratio of H3:H4 is 1-5:1, the weighted ratio of H1:H2 is 1:0.4-2, the weighted ratio of H1:H4 is 1:6.5-8, and the weighted ratio of H1:H5 is 1:1.5-2 and is referred to as H12345 formulation thereafter.

### Example 2. Evaluation of Anticancer Activity of Individual Herbal Extracts and Herbal Formulations In Vitro

DLD-1 and HCT1116 were the chosen cell lines used in this study. Both cell lines were maintained as per specific requirement for each line. Both cell lines were maintained in RPMI 1640 medium supplemented with 10% FBS and 1% Penicillin/Streptomycin. Herbal extracts in 100 mg/ml stock concentrations were diluted with RPMI 1640 cell culture media whenever necessary.

### Cell Viability Assay

The dose dependent effect of the individual herbal extracts and formulations on cell viability of the colon cancer cell lines were tested using MTT assay. 11,000 cells were seeded onto a 96-well tissue culture plate and treated for 48 hours with increasing concentrations of the herbal extracts. 10 μl of MTT (5 mg/ml) was added to 100 μl media in each well and incubated for 4 hours. Purple formazan crystals derived from the reduction of MTT by metabolically active cells were dissolved in DMSO and absorbance measured at 570 nm using a microplate spectrophotometer (Bio-Rad).

### Clonogenic Assay

The colongenic ability of DLD-1 cells after herb extracts treatment was assessed using a colognenic assay. Single cells were treated with either LD (0.01 mg/ml), MD (0.025 mg/ml) or HD (0.05 mg/ml) of the single herb extracts or herbal formulation for 1 hour and allowed to continue growing for 7 days to form colonies. Colonies were fixed and stained with 0.5% w/v crystal violet, then rinsed with distilled water before scanning for quantification. Colony counts and area was quantified using Image J software.

### Tumor Spheroid Growth Assay

A tumor spheroid assay was performed to assess the anti-tumorigenic potential of herb treatment on DLD-1 viability within a simulated tumor microenvironment in vitro. Briefly, a small tumor spheroid was generated by seeding 8000 cells on an agarose-coated 96-well tissue culture plate followed by centrifugation at 800 g for 5 minutes. The spheroids were then allowed to grow with or without herb treatments for 15 days. 50% media replacement with or without the herb extracts was carried out every 3 days. Phase contrast images of the spheroids were taken every other day and the area of the spheroids were quantified using Fiji software.

### Morphological Changes by Live Cell Imaging

DLD-1 cells were seeded on a 24-well tissue culture plate, treated with individual herb extracts or herbal formulations H34 and H12345 at LD (0.01 mg/ml), MD (0.025 mg/ml) and HD (0.05 mg/ml) concentrations and placed on a heat-controlled stage of a Zeiss Axiovert 200M microscope to study cell morphological changes and cell fates. Phase contrast images were acquired at 15 minutes intervals for 72 hours at 37° C. and 5% CO₂ levels.

### Cell Cycle Distribution and Apoptosis

Cell cycle distribution of DLD-1 cells treated with H3, H34 and H12345 was investigated by flow cytometry. DLD-1 cells were harvested 24 hours after treatment with the extracts and fixed with 70% ethanol. The cells were washed and suspended in PBS containing propidium iodide and RNAase for 20 minutes. Cell cycle distribution was recorded using a three-laser LSRFortessa™ X-20 Cell equipped with a FACSDiva™ software. The cytometry data was analysed using FlowJo software.

### Western Blotting

The proteins were separated by Sodium Dodecyl Sulphate Polyacrylamide Gel Electrophoresis (SDS-PAGE) at 120 V for 90 minutes using 12% or 15% polyacrylamide gels. 20 μg of proteins were loaded into the wells of the gels. Resolved proteins were transferred onto 0.45 μm PVDF membranes at 100 V for 80 minutes. Membrane blocking was performed for 1 hour at room temperature with 5% (w/v) skimmed milk in 1× tris-buffered saline with Tween 20 (TBST). The membranes were probed with primary antibodies at 4° C. overnight, followed by incubation with the appropriate HRP-conjugated secondary antibodies for 1 hour at room temperature. All antibody incubations were performed in 5% milk in 1×TBST, and the membranes were washed thrice for 10 minutes in 1×TBST after each antibody probing step. The WesternBright™ ECL HRP Substrate kit was used for detection of chemiluminescent signals. The blots were developed for band visualisation using the Bio-Rad ChemiDoc™ MP Imaging System and processed with Image Lab Version 6.1.0 (Bio-Rad, USA).

### Results and Discussion

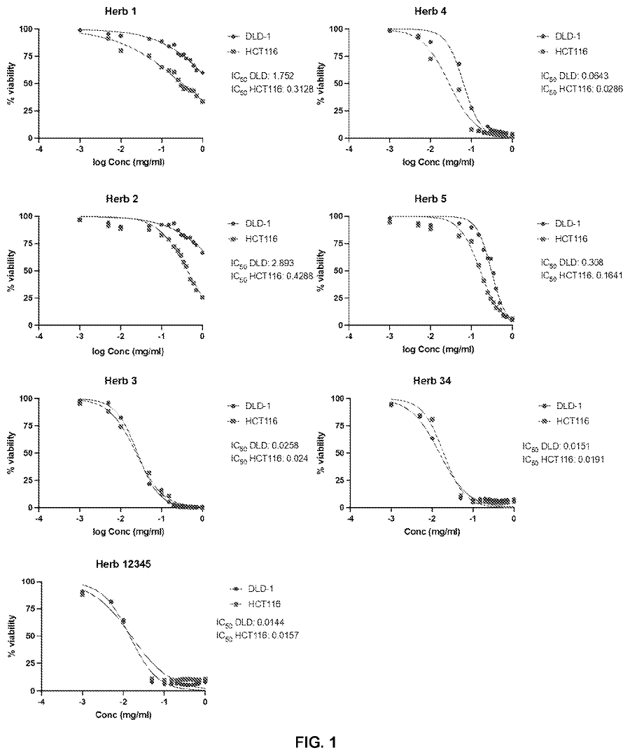

Absolute IC₅₀ values of the individual herb extracts and the two herbal formulations were calculated (Table 1, FIG. 1). It was found that H3 and H4 were very effective in inhibiting the proliferation of the colon cancer cells. The IC₅₀ values for these 2 herbs are significantly lower than the rest. Combining the herbs into formulation H34 and H12345 could further lower the IC₅₀ value, suggesting stronger efficacy in inhibiting cancer cell growth.

TABLE 1
IC₅₀ of individual herb extracts and herbal
formulation against colon cancer cell lines.

| | |
|---|---|
| DLD-1 | HCT116 |

| | | | | |
|---|---|---|---|---|
| IC₅₀ H1 | 1.752 | mg/ml | 0.3128 | mg/ml |
| IC₅₀ H2 | 2.893 | mg/ml | 0.4288 | mg/ml |
| IC₅₀ H3 | 0.0258 | mg/ml | 0.024 | mg/ml |
| IC₅₀ H4 | 0.0643 | mg/ml | 0.0286 | mg/ml |
| IC₅₀ H5 | 0.308 | mg/ml | 0.1641 | mg/ml |
| IC₅₀ H34 | 0.0151 | mg/ml | 0.0191 | mg/ml |
| IC₅₀ H12345 | 0.0144 | mg/ml | 0.0157 | mg/ml |

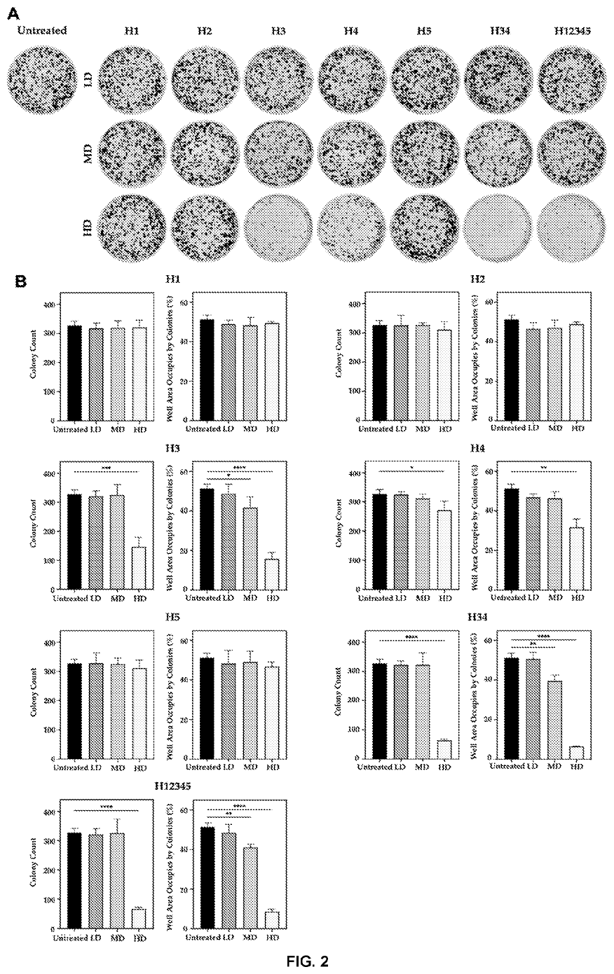

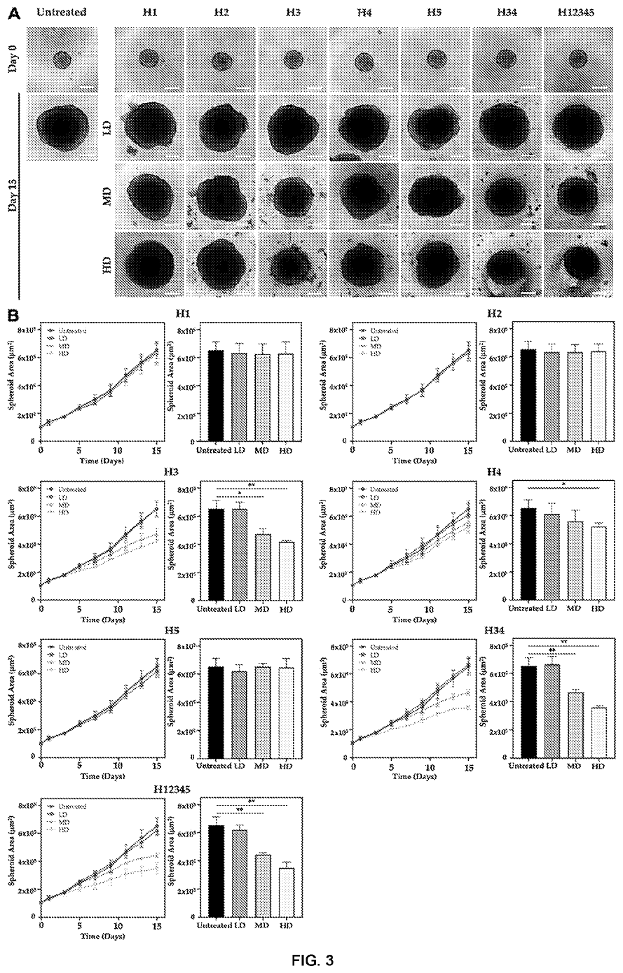

It was found that H3 and herbal formulation H34 and H12345 significantly impacted DLD-1 clonogencity. While control, H1, H2 and H5 treatment groups resulted in colony counts above 300 and covering 45% of the well area across all concentrations tested, HD concentrations of H3 greatly reduced colony counts to under 150 colonies that covered 16% of the well's surface. Combining H3 with other herbs into herbal formulation H34 and H12345 further reduced the colony counts to about 60 colonies, covering less than 10% of the well area. The results indicate that H3 and the formulations H34 and H12345 are able to effectively inhibit the proliferation of DLD-1 cells (FIG. 2, Table 2).

TABLE 2
Mean ± SD Colony counts and percentage of well area occupied
by DLD-1 colonies by treatment group and dose (n = 3).

| | | | Well Area Occupied by |
|---|---|---|---|
| Treatment | Dose | Colony Count | Colonies (%) |
| Untreated (Control) | — | 327.33 ± 15.18 | 51.31 ± 2.20 |
| Herb 1 | LD | 317.00 ± 18.25 | 48.92 ± 2.05 |
| | MD | 319.67 ± 24.01 | 48.32 ± 3.92 |
| | HD | 321.00 ± 24.76 | 49.38 ± 0.95 |
| Herb 2 | LD | 326.00 ± 34.39 | 46.53 ± 3.05 |
| | MD | 327.00 ± 7.21 | 47.05 ± 3.97 |
| | HD | 310.67 ± 27.57 | 48.88 ± 1.09 |
| Herb 3 | LD | 320.33 ± 19.01 | 48.70 ± 4.77 |
| | MD | 325.00 ± 36.00 | 41.53 ± 5.60 |
| | HD | 146.33 ± 32.88 | 15.80 ± 3.24 |
| Herb 4 | LD | 325.00 ± 9.85 | 46.82 ± 1.80 |
| | MD | 312.00 ± 15.10 | 46.41 ± 3.40 |
| | HD | 272.00 ± 30.51 | 31.67 ± 4.30 |
| Herb 5 | LD | 328.33 ± 35.36 | 48.33 ± 6.66 |
| | MD | 325.33 ± 20.53 | 49.10 ± 5.53 |
| | HD | 311.33 ± 27.47 | 46.75 ± 2.29 |
| Herbs 34 | LD | 322.67 ± 13.01 | 50.77 ± 3.31 |
| | MD | 323.33 ± 40.92 | 39.50 ± 3.05 |
| | HD | 63.67 ± 5.03 | 6.43 ± 0.15 |
| Herbs 12345 | LD | 322.00 ± 19.92 | 48.47 ± 4.23 |
| | MD | 326.33 ± 46.93 | 41.05 ± 1.79 |
| | HD | 66.33 ± 5.51 | 8.43 ± 1.40 |

The tumor spheroid growth assay results showed that spheroids treated with H3, H4, H34 and H12345 extracts resulted in a stunted growth of the spheroids in a dose dependent manner while H1, H2 and H5 extracts have minimal effect on spheroid growth as compared to control untreated spheroids (FIG. 3).

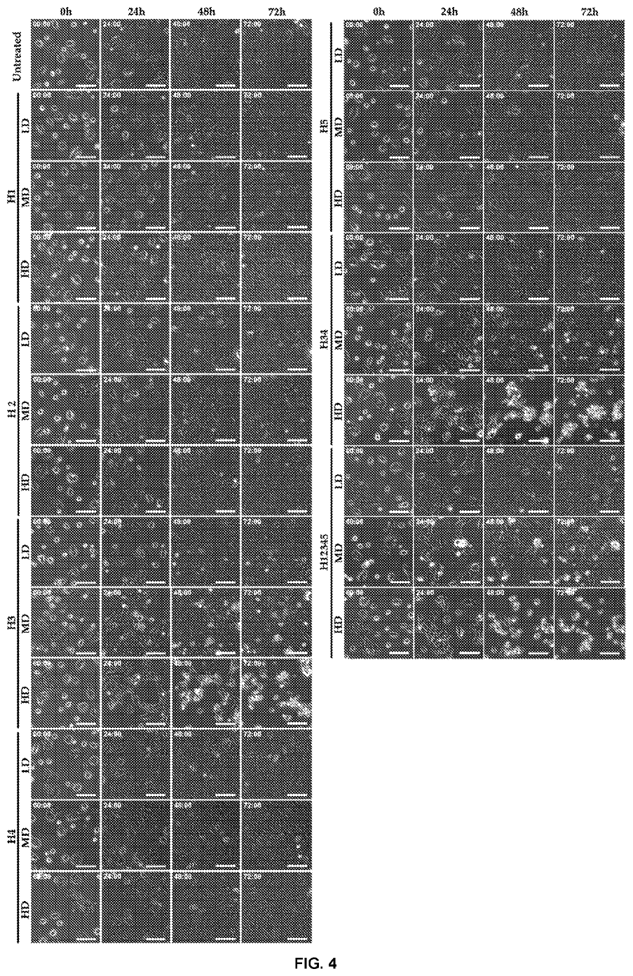

While untreated cells as well as H1, H2, H4 and H5 extracts-treated cells were able to grow and proliferate throughout the course of live imaging, DLD-1 cells treated with H3 and the formulations H34 and H12345 exhibited cell rounding, mitotic arrest, detachment from the substrata and eventually cell death in a dose dependent manner (FIG. 4).

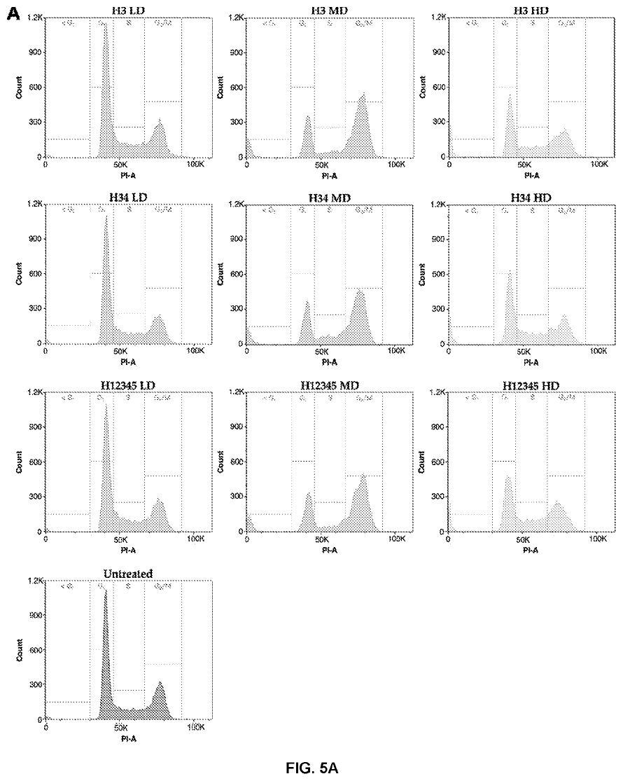

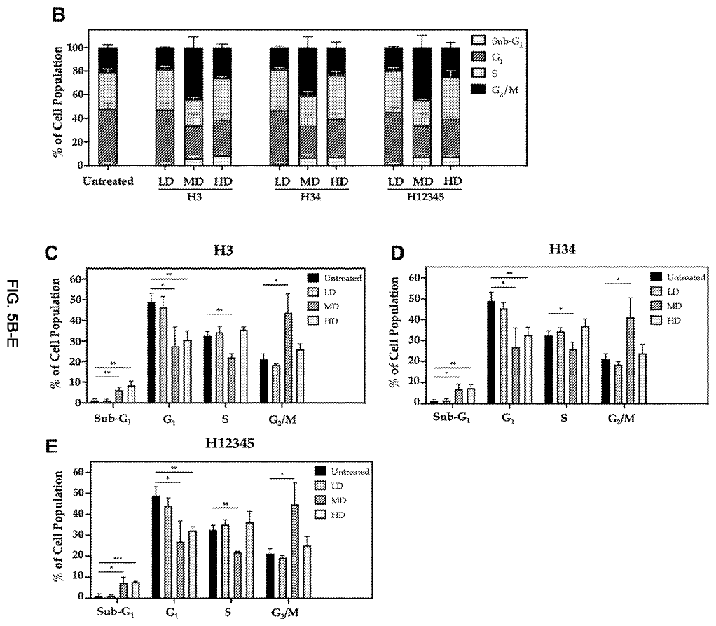

It was found that there were significant differences in the cell cycle stages between high concentrations of herb extract treatment compared to control untreated cells. MD and HD herb treatment groups had a significantly higher percentage of cells in sub G1 stage, indicating the presence of apoptotic cells. Additionally, there was also a profound increase in the G2/M cell population at MD doses of herb treatment, indicating that the herb treatment caused a cell cycle arrest at the G2/M phase (FIG. 5).

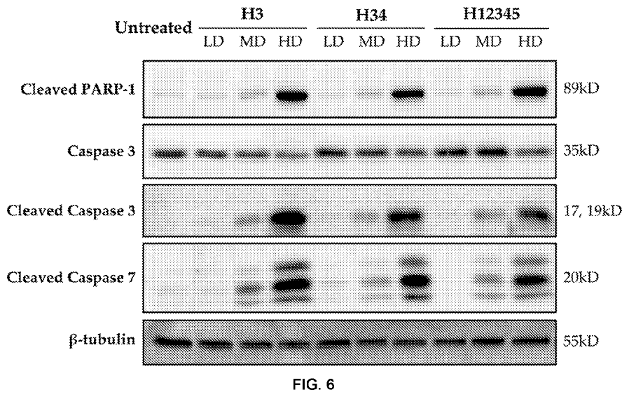

To investigate if the herb extracts cause cell death by apoptosis, the expression of pro-apoptotic markers was quantified by western blotting following a 24-hours herb treatment of DLD-1 cells. Compared with untreated cells, the herb-treated cells displayed elevated cleaved form of caspase-3, -7 and PARP1 in a dose dependent manner. The results suggest that cell death induced by the herb extracts involves the apoptotic pathway (FIG. 6).

### Example 3. Evaluation of Herbal Formulations on Tumour Cell Growth In Vivo

### In Vivo Studies

Five to six-weeks old female J:Nu outbred nude mice were purchased from InVivos with body weight around 20-23 g. The mice were allowed to acclimate for at least 5 days before they were subjected to subcutaneous injection with 1×10⁶ HCT116 or DLD-1 cells to create xenograft models. The mice were randomly divided into 2 groups, control versus treatment groups. Mice in the control and treatment groups were fed with water and herbal formulations respectively, twice daily by oral gavage for a duration of up to 4 weeks. The mice were administered with 424 mg/kg (amount of material in the solvent) dose and 536 mg/kg (amount of material in the solvent) dose of the H34 and H12345 formulations, respectively. The ratio of H3:H4 in the H34 formulation is 1.65:1 and the ratio of H1:H2:H3:H4:H5 in the H12345 formulation is 1.7:1:22:13.3:6.7. Tumour volumes were measured twice a week. Tumour volumes were calculated using the formula (L×W²)/2, where L and W are the length and width of the tumours, respectively. Inhibition of tumour growth was expressed as the (T/C %) ratio, the ratio of the median tumour volume for the treated vs control group. All procedures were approved by NTU Institutional Animal Care and Use Committee (IACUC A20001).

### Results and Discussion

The herbal formulations H34 and H12345 can inhibit HCT116 and DLD-1 tumour cell growth in nude mice.

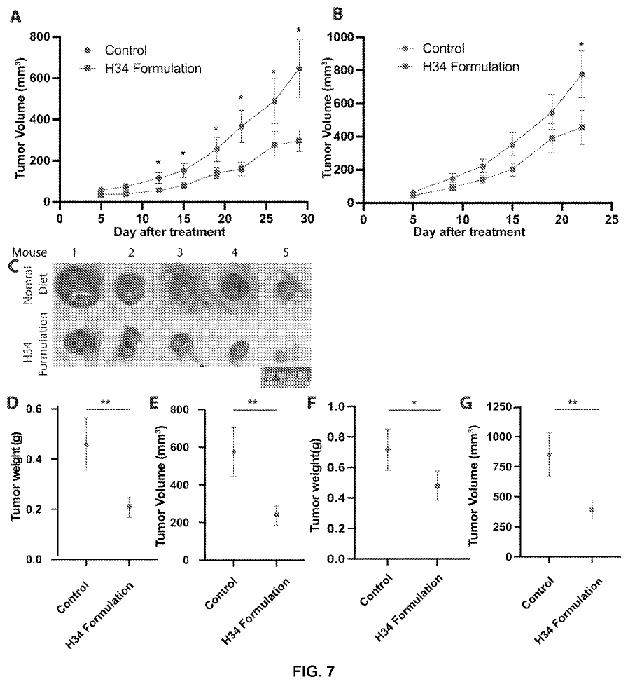

Treatment group mice that were orally administered with H34 herbal extracts exhibited a significantly smaller tumour cell growth for both HCT116 and DLD-1 xenografted mice (FIG. 7). Average tumour volumes for control (n=18) and treatment (n=18) were 575.8 mm³ and 249.8 mm³, respectively (p<0.05, 1 tail T-test, 95% confidence). The average tumour weights for control (n=18) and treatment (n=18) were 0.45 g and 0.20 g, respectively, for the HCT116 xenografted mice (p<0.05, 1 tail T-test, 95% confidence). For DLD-1 xenografted mice, the average tumour volumes for control (n=17) and treatment (n=17) were 850.7 mm³ and 394.6 mm³ (p<0.05, 1 tail T-test, 95% confidence), respectively, and average tumour weights were 0.72 g and 0.48 g (p<0.1, 1 tail T-test, 90% confidence), respectively. Significant antitumor activity was obtained for the H34 treated HCT116 and DLD-1 xenografted mice with a T/C % of 43.3% and 46.4% (p<0.05, 1 tail T-test, 95% confidence), respectively (FIG. 7).

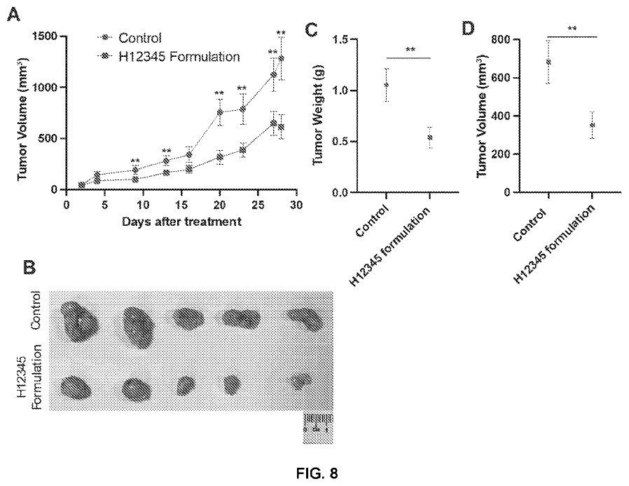

The effect of herbal formulation H12345 on tumour cell growth in nude mice was also evaluated. The results were similar to mice treated with H34 formulation. HCT116 xenografted mice displayed significantly smaller tumour cell growth when orally administered with H12345 formulation. Average tumour volumes for control (n=14) and treatment (n=13) were 1071 mm³ and 515.8 mm³, respectively, and average tumour weights were 1.05 g and 0.54 g, respectively. Both parameters measured were statistically significant between the control and treatment groups (p<0.05, 1 tail T-test, 95% confidence). The T/C % ratio is 48.1%. There were no drastic differences in the body weight of the mice between control and treatment groups throughout the course of treatment (FIG. 8).

Our herbal formulations, H34 and H12345, have showed that they are effective in inhibiting the cell viability of colon cancer cells in vitro and also demonstrated significant anti-tumour cell growth in vivo. There is a strong potential that these formulations can be used as a medicine or supplement for colon cancer treatment, prevention or serve as an adjuvant therapy for standard treatment.

1. A herbal formulation comprising a herbal extract of Elephanopus tomentosus L and a herbal extract of Leea indica.

2. The herbal formulation according to claim 1, wherein the formulation comprises one or more of:
an extract of Clinacanthus nutans;
an extract of Strobilanthes crispus L.; and
an extract of Callicarpa pedunculata R. Br.

3. The herbal formulation according to claim 1, wherein the formulation consists of the herbal extract of Elephanopus tomentosus L and the herbal extract of Leea indica.

4. The herbal formulation according to claim 1, wherein the weight to weight ratio of the herbal extract of Elephanopus tomentosus L and the herbal extract of Leea indica is from 1:1 to 5:1, such as about 1.7:1

5. The herbal formulation according to claim 2, wherein the extract of Clinacanthus nutans is present.

6. The herbal formulation according to claim 5, wherein the extract of Strobilanthes crispus L. is also present.

7. The herbal formulation according to claim 6, wherein the weight to weight ratio of the extract of Clinacanthus nutans and the extract of Strobilanthes crispus L. is from 1:0.4 to 1:2.

8. The herbal formulation according to claim 5, wherein the weight to weight ratio of the extract of Clinacanthus nutans and the extract of Elephanopus tomentosus L. is from 1:1 to 1:11.

9. The herbal formulation according to claim 5, wherein the weight to weight ratio of the extract of Clinacanthus nutans and the extract of Leea indica is from 1:6.5 to 1:8.

10. The herbal formulation according to claim 5, wherein the extract of Callicarpa pedunculata R. Br. is also present.

11. The herbal formulation according to claim 6, wherein the weight to weight ratio of the extract of Clinacanthus nutans and the extract of Callicarpa pedunculata R. Br. is from 1:1.5 to 1:2.

12. The herbal formulation according to claim 2, wherein the formulation comprises:
the extract of Clinacanthus nutans;
the extract of Strobilanthes crispus L.;
the extract of Elephanopus tomentosus L.;
the extract of Leea indica; and
the extract of Callicarpa pedunculata R. Br.

13. The herbal formulation according to claim 12, wherein the weight to weight ratio of the extract of Clinacanthus nutans to the extract of Strobilanthes crispus L. to the extract of Elephanopus tomentosus L.to the extract of Leea indica to the extract of Callicarpa pedunculata R. Br. is 2:1:22:13:4.

14. (canceled)

15. A method of preparing a herbal formulation, comprising:
(a) providing stock solutions of:
an extract of Elephanopus tomentosus L.; and
an extract of Leea indica; and
(b) combining a portion of the stock solution of the extract of Elephanopus tomentosus L. with a portion of the stock solution of the extract of Leea indica to provide the herbal formulation.

16. The method according to claim 15, wherein the method further comprises:
(i) further providing stock solutions of:
an extract of Clinacanthus nutans;
an extract of Strobilanthes crispus L.; and
an extract of Callicarpa pedunculata R. Br; and
(ii) combining a portion of one or more of the extract of Clinacanthus nutans, the extract of Strobilanthes crispus L. and the extract of Callicarpa pedunculata R. Br in step (b) of the method according to claim 15.

17. The method according to claim 15, wherein each extract is prepared by
(ai) providing a powder of a freeze-dried herb;
(aii) mixing the powder of a freeze-dried herb with a first solvent to provide a mixture and subjecting the mixture to sonication for a period of time to provide a sonicated mixture;
(aiii) removing solids from the sonicated mixture by centrifugation to collect a soluble portion of the sonicated mixture; and
(aiv) removing the solvent from the soluble portion and then adding a second solvent to provide a stock solution, optionally wherein the stock solution has a concentration of from 50 to 200 mg/mL, such as about 100 mg/mL

18. (canceled)

19. (canceled)

20. A method of preparing a herbal formulation, comprising:
(bi) providing a powder of two of more freeze-dried herbs;
(bii) mixing the powder of two or more freeze-dried herbs with a first solvent to provide a mixture and subjecting the mixture to sonication for a period of time to provide a sonicated mixture;
(biii) removing solids from the sonicated mixture by centrifugation to collect a soluble portion of the sonicated mixture as the herbal formulation, wherein
the two or more herbs comprise Elephanopus tomentosus L. and Leea indica.

21. The method according to claim 20, wherein the two or more herbs further comprise one or more of Clinacanthus nutans, Strobilanthes crispus L. and Callicarpa pedunculata R. Br.

22. (canceled)

23. (canceled)

24. A pharmaceutical composition comprising a herbal formulation as defined in claim 1 and a pharmaceutically acceptable excipient.

25. (canceled)

26. (canceled)

27. A method of treating cancer, which method comprises administering a therapeutically effective amount of a herbal formulation as defined in claim 1 to a subject in need thereof.

28. The method according to claim 27, wherein the cancer is selected from one or more of the group selected from adrenal cancer, anal cancer, bile duct cancer, bladder cancer, bone cancer, brain tumours, CNS tumours, breast cancer, Castleman disease, cervical cancer, colon cancer, rectum cancer, colorectal cancer, endometrial cancer, esophagus cancer, eye cancer, gallbladder cancer, gastrointestinal carcinoid tumors, gastric cancer, gastrointestinal stromal tumor (GIST), gestational trophoblastic disease, Hodgkin disease, Kaposi sarcoma, kidney cancer, laryngeal cancer, hypopharyngeal cancer, leukemia (e.g. acute lymphocytic, acute myeloid, chronic lymphocytic, chronic myeloid, chronic myelomonocytic), liver cancer, lung cancer (e.g. small cell or non-small cell), lung carcinoid tumour, lymphoma (e.g. of the skin), malignant mesothelioma, multiple myeloma, myelodysplastic syndrome, nasal cavity cancer, paranasal sinus cancer, nasopharyngeal cancer, neuroblastoma, non-Hodgkin lymphoma, oral cavity cancer, oropharyngeal cancer, osteosarcoma, ovarian cancer, pancreatic cancer, penile cancer, pituitary tumours, prostate cancer, retinoblastoma, rhabdomyosarcoma, salivary gland cancer, sarcoma, skin cancer (basal and squamous cell, melanoma, Merkel cell), small intestine cancer, stomach cancer, testicular cancer, thymus cancer, thyroid cancer, uterine sarcoma, vaginal cancer, vulvar cancer, Waldenstrom macroglobulinemia, Wilms tumour.

29. (canceled)
